(19) United States
(12) Patent Application Publication
(10) Pub. No.: US 2011/0265795 A1
(43) Pub. Date: Nov. 3, 2011

(54) MAINTENANCE AND MANAGEMENT SYSTEM FOR ENDOTRACHEAL TUBE IN INTUBATION

(75) Inventors: Yoshihiko Tagawa, Kitakyusyu-shi (JP); Koji Mimura, Kitakyusyu-shi (JP); Tomohisa Inada, Kitakyusyu-shi (JP); Takahiro Matsumoto, Kitakyusyu-shi (JP)

(73) Assignee: KYUSYU INSTITUTE OF TECHNOLOGY, Kitakyusyu-shi (JP)

(21) Appl. No.: 12/891,370

(22) Filed: Sep. 27, 2010

Related U.S. Application Data
(63) Continuation-in-part of application No. PCT/JP2009/055453, filed on Mar. 19, 2009.

(30) Foreign Application Priority Data
Mar. 26, 2008 (JP) 2008-080385

(51) Int. Cl. A61M 16/04

(52) U.S. Cl. 128/205.23

(57) ABSTRACT

A maintenance and management system for tracheal tube in tracheal intubation makes it easier or steadier to identify endotracheal tube placement in the airway, check a tracheal stenosis due to sputum clogging, confirm a breakaway of the tube out of the airway, store and monitor measured data, diagnose automatically the complications, and obtain data for reproducing function of sound in a breath circuit. A ventilator tube is connected to the endotracheal tube and provided therein with a microphone to sense a breath sound transmitted via the endotracheal tube and a pressure sensing element to measure a circuit pressure in the ventilator circuit for the endotracheal tube. The data derived from the sensing elements is processed in a personal computer to be used to measure whether the breath sound and the circuit pressure are within a range of a prescribed value.

### FIELD OF THE INVENTION

The present invention relates generally to a tracheal intubation and more particularly to a system to maintain or keep in place and manage or administer adequately an endotracheal tube after a trachea or an airway has been intubated for mechanical ventilation at, for example, medical practice sites of anesthesia, intensive care, first-aid care and so on.

### BACKGROUND AND REVIEW OF RELATED TECHNOLOGY

The tracheal intubation is generally a standard medical care in which an endotracheal tube is inserted or introduced for mechanical ventilation into a trachea of a patient who has no airway management and then no sufficient ventilation and no oxygenation of blood at medical practice sites of anesthesia, intensive care, first-aid care and so on. With the tracheal intubation as stated earlier, the trachea is usually intubated with the endotracheal tube while a laryngoscope makes a view of the vocal folds and the glottis possible. The endotracheal tube in the tracheal intubation, although normally advanced into the trachea through an oral cavity or a mouth, can be inserted through the nose, especially at the time when the patient has an operation on his mouth. Moreover, some complications caused from the tracheal intubation can be foreseen, prevented, treated and cured by virtue of continuous watch and monitoring by doctors and nurses. Nevertheless, many people would suffer fatal risks of postoperative complications if discovered too late. The above is why the endotracheal tube maintenance and management are critical.

Meanwhile, the tracheal intubation is a medical technology which needs highly advanced techniques and a great deal of clinical experience. Different types of laryngoscope systems for the tracheal intubation have been developed to make insertion of the tracheal tube into the trachea easier and safer. Disclosed in, for example Japanese Laid-Open Patent Application No. 2006-149 450 is one of the prior laryngoscope systems for the tracheal intubation, which is motivated to make it easier than ever to give an operator a sight or view of operating procedure, making the manipulation of the laryngoscope safer with more certainty, and thus making for clinical experience or clinical status as well as medical education or training. The prior laryngoscope system is composed of a laryngoscope handle having mounted therein a gyroscope, and a blade having a strain gauge or sensing element. Upon intubation while using the laryngoscope system, the strain gauge detects a load exerted on the blade from the teeth and the gyroscope senses an angular velocity and a moving direction caused by the handle operation. Any warning sign is issued after any of the detected load on the blade and the sensed movement of the handle has been beyond their preselected critical levels.

Another Japanese Laid-Open Patent Application No. H09-187 510 discloses a cuff-pressure regulator for a cuffed endotracheal tube, which is able to shrink in size because no use of the electric power source. With the prior cuff-pressure regulator, too much of the air can be released out of the cuff whenever the cuff pressure rose not only at the time of air pouring phase but also even under medical care. The cuff-pressure regulator for the cuffed endotracheal tube of the type as stated earlier is composed of a cuff-pressure regulator body having a container in which there are provided an air inlet communicated with a pneumatic resource, an outlet for air to be delivered into an air tube connected to a cuff, and a window lying midway between the air inlet and outlet. The cuff-pressure regulator body further includes a closure to normally seal the window, but actuated to open the window after the pneumatic pressure inside the regulator body have risen up to a preselected threshold level, and a check valve having a valve seat made of a partition holed near the air inlet inside the regulator body.

### DISCLOSURE OF THE INVENTION

### Technical Problems to be Challened

While the tracheal intubation is a standard medical procedure for the airway management and the administered mechanical ventilation, the medical education and training of the tracheal intubation are usually performed with the help of medical simulation mannequins, models or related artifacts. Of the clinical experiences on physical examination, clinical test and manual therapy the residents are required to reach desired objectives in the clinical training for residents, there is the practical skill on the tracheal intubation. Thus, the residents are normally required to have undergone the training on the airway management over a predetermined period in the fields of anesthesia and first-aid care. Moreover, the medical technicians or paramedics are licensed to perform the tracheal intubation at the emergency sites after the completion of required course at the fire fighter's college and 30 successes in the tracheal intubations under the guidance of anesthesiologists. On actual tracheal intubation, there are considered the associated complications recited later.

1. Unrecognized esophageal intubation: the endotracheal tube is not introduced into the trachea, but wrongly into the esophagus.

2. Obstruction of the endotracheal tube caused by secreted chemical substance, sputum and collapse or bent in the endotracheal tube, etc.

3. The endotracheal tube falls away from the trachea because of some reason especially after prolonged tracheal intubation, consequently resulting in aberrant esophageal intubation or extubation.

4. Aberrant aspiration of foreign body such as digestive juice, blood and so on caused by air leakage resulting from the cuff-pressure reduction inside the endotracheal tube.

5. Ischemia and damage of the tracheal mucosa caused by the oppressive pressure occurring in the blood flow owing to excessive cuff pressure in the endotracheal tube.

The concerns of the associated complications as recited just earlier would be foreseen or found, treated and cured by virtue of the advanced medical equipment and medical diagnosis. Nevertheless, if the correct diagnosis is too late, the patients would suffer fatal risks. Thus, it is of critical importance to sustain the endotracheal tube maintenance and management, still after the trachea has been completely intubated with the endotracheal tube without negligence. So far, there is no disclosure or teaching about a diagnosis method of attempting simultaneously to evaluate conditions of the endotracheal tube and to detect the esophageal intubation with using just a single system at the time of the mechanical ventilation. Although but there is known the prior technology to regulate the cuff pressure, knowing the resultant cuff pressure was optimal one was a challenge. While the tracheal intubation is a common medical procedure in present-day hospitals, the systems for monitoring their patients vary. For instance, the tracheal intubation in the operating room is considered to be comparatively safe because of monitored over a sustained period of time under the supervision of the anesthesiologists, and the intensive care unit is also considered highly safe because of under the professionally managed or administered monitoring system. As opposed to the above, many of the ordinary wards aren't necessarily better in monitoring system at the time of mechanical ventilation. Thus, it remains a major challenge to develop a system making it possible to manage the endotracheal tube in unremitting way.

The present invention is envisaged measuring values or quantities representing conditions of an endotracheal tube using the existing sensing technology to do appropriately the maintenance and management of the endotracheal tube, thereby previously keeping associated complications from occurring. One of the big challenges in the tracheal intubation was how treating or processing signals outputted from a sensing element installed in the system to reduce the interference from nearby or surrounding noises and so on. The tracheal intubation would be considered to be needed increasingly more frequently in the period to come. However, the present-day tracheal intubation has a problem or issue as stated later. The clinicians as a matter of fact rely on auscultation of the breath sounds with the help of the stethoscope for diagnosis of modest airway obstruction. This diagnosis, even though convenient and easy, is in need of a great deal of skill or clinical experience because vulnerable to varieties of the breath sounds and impacts of nearby noises. Moreover, there is no quantified criterion to learn how to evaluate the auscultation of the breath sound.

So far there is no provision of a method and/or equipment for certainly diagnosing conditions of the tracheal intubation in every situation and/or confirming the esophageal intubation with the aid of various existing instruments, technologies and combination thereof. Such diagnosis technique, if any, would be unavailable instantly or easily in some situations of the emergency rooms and hospitals. To cope with this, the present inventors have developed a system smaller in size and inexpensive in production to maintain or keep in place and manage or administer adequately an endotracheal tube, which is provided by only the unique combination of usual commercial components rather than using any novel electronics and/ or custom-built products.

### SUMMARY OF THE INVENTION

The present invention intended to overcome the problems stated earlier has as its principal object the provision of a system in which an existing sensing element, for example a microphone senses breath sounds in a breathing circuit while a pressure sensing element detects a pressure in the breathing circuit, and information issued from the sensing elements is available to early detect and warn of the complications lying in modest status. The present invention has as more particular object to provide a maintenance and management system for an endotracheal tube inclusive of an automatic regulation of a cuff pressure which isn't automated so far in management of mechanical ventilation. With the system constructed according to the present invention, an estimated condition of the endotracheal tube makes earlier treatment or procedure easier to assist the clinical experience and medical education. Moreover, signals are treated to get the estimated condition of the endotracheal tube greater in accuracy and noises mixed in the information are made less. The existing sensing elements and actuators are used as ancillary means to carry out the automatic regulation of the cuff pressure as well as the estimate of the cuff pressure considered optimal one to the patient, making it possible to find not only the complications which have been diagnosed with conventional devices, but also the airway obstruction lying in modest status which would be tough to find with conventional systems.

The present invention relates to a maintenance and management system for tracheal tube comprising; an endotracheal tube for airway management, an air supply system to force air into a desired area through the endotracheal tube, a tube connecting the endotracheal tube with the air supply system to complete a ventilator circuit, a microphone to sense a breath sound transmitted via the endotracheal tube, a first pressure sensing element to measure a circuit pressure in the ventilator circuit, the microphone and first pressure sensing element being built in the ventilator circuit tube, a personal computer receiving data derived from the microphone and first pressure sensing element and processing the data therein, the personal computer measuring whether the breath sound sensed with microphone is within a desired value range and then signing the data if within the desired value range while issuing abnormality output signal instead of the data if over the desired value range, and the personal computer further measuring whether the circuit pressure detected with the first pressure sensing element is within a desired value range and then signing the data if within the desired value range while issuing abnormality output signal instead of the data if over the desired value range;

an adaptive noise canceller comparing a noise other than the breath sound sensed with the microphone to a signal detected with an outer microphone installed nearby the microphone and outside of the ventilator circuit, then excluding the noise; and

an adaptive line enhancer extracting a narrowband signal from a broadband signal which includes only the breath sound outputted from the adaptive noise canceller.

In the present invention, there is disclosed a maintenance and management system for tracheal tube, which further has a low-pass filter to exclude a noise other than the circuit pressure included in a signal sensed with the first pressure sensing element.

In aspect of the present invention, there is disclosed a maintenance and management system for tracheal tube in which the personal computer, when the data detected with the microphone and the first pressure sensing element correspond to the abnormality output signal, measures what complication is taught based on the data, displaying and warning the complication.

In the present invention, there is disclosed a maintenance and management system for tracheal tube in which the personal computer measures and warns the complication to be an air leak occurring in the endotracheal tube, in response to an abnormal frequency power of the breath sound after noise reduction of the signal derived from the microphone.

In the present invention, there is disclosed a maintenance and management system for tracheal tube in which the personal computer measures and warns the complication to be a stenosis occurring in the endotracheal tube, in response to an abnormal frequency power of the breath sound after signal processing of the signal derived from the microphone.

In the present invention, there is disclosed a maintenance and management system for tracheal tube, which further has a cuff-pressure regulator connected to the endotracheal tube through a pilot balloon to regulate a cuff pressure in a cuff attached to the endotracheal tube, and a second pressure sensing element to gauge the cuff pressure, and wherein the cuff-pressure regulator controls a pneumatic pressure applied to the cuff in response to a data originating in the second pressure sensing element, keeping and regulating the cuff pressure at a prescribed pressure.

In the present invention, there is disclosed a maintenance and management system for tracheal tube in which the personal computer measures whether the cuff pressure gauged with the second pressure sensing element is in normal state or abnormal state, keeping the state detected with the second pressure sensing element if in normal state, and instead warning occurrence of the complication if in abnormal state.

In the present invention, there is disclosed a maintenance and management system for tracheal tube in which the personal computer in response to the abnormal cuff-pressure gauged with the second pressure sensing element measures and warns the complication to be an air leak caused by a cuff-pressure reduction, aberrant aspiration of foreign body including digestive juice, blood and so on into the airway, or an oppressive pressure against a blood flow in a tracheal mucosa.

### Advantageous Effects of the Invention

A maintenance and management system for a tracheal tube constructed according to the present invention excels in permitting diagnosis of not only the complications which could be measured with even conventional systems, but also the modest airway obstruction which was hard to be found by the traditional systems. The system of the present invention, moreover, is excellent in permitting providing the optimal cuff-pressure estimate customized for every patient rather than providing only the automatic regulation of the cuff pressure, which has been carried out with conventional systems.

The system constructed according to the present invention has different advantageous effects, for example, checking whether the endotracheal tube after having passed through the glottis is placed properly in the trachea, not in the esophagus, confirming no occurrence of the stenosis in the endotracheal tube caused by sputum clogging, checking whether the endotracheal tube is falling away from the airway, confirming there is no air leak through the airway, permitting storage and monitoring of measured data, automatic diagnosis of the associated complications, and further taking information about reproducing function of sound in the breathing circuit, and so on.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic view showing a preferred embodiment of a maintenance and management system for an endotracheal tube according to the present invention:

FIG. 2 is an illustration explanatory of a device for sensing and measuring a breath sound and a circuit pressure, and a cuff-pressure regulator:

FIG. 3 is a flow diagram illustrating a signal processing procedure in the device for sensing and measuring the breath sound and circuit pressure:

FIG. 4 is a graphic representation of pressure variations in a breathing circuit:

FIG. 5 is a block diagram showing an adaptive noise canceller:

FIG. 6 is a graphic representation illustrating waveform patterns of a breath sound with no mixed noise detected with a microphone:

FIG. 7 is a graphic representation showing waveform patterns a breath sound mixed with noise detected with a microphone:

FIG. 8 is a block diagram showing an adaptive line enhancer:

FIG. 9 is a graphic representation showing a sinusoidal signal, random signal and a signal resulting from superposition of the random signal on the sinusoidal signal, and the results of their time-frequency analyses:

FIG. 10 is a graphic representation showing a signal resulting from superposition of the random signal on the sinusoidal signal to be input to an adaptive line enhancer, and an output signal and an error signal which are derived from the adaptive line enhancer along with the results of their time-frequency analyses:

FIG. 11 is a graphic representation showing samples of data derived from a pressure sensor and two microphones:

FIG. 12 is a graphic representation showing signal patterns of the data in FIG. 11 after subjected to signal processing in a phase the endotracheal tube remains in normal condition:

FIG. 13 is a graphic representation showing signal patterns of the data in FIG. 11 after subjected to signal processing in a phase the endotracheal tube gets partially collapsed:

FIG. 14 is a graphic representation showing signal patterns of the data in FIG. 11 after subjected to signal processing in a phase a reduction in the cuff pressure causes any air leakage:

FIG. 15 is a graphic representation showing other samples of data derived from a pressure sensor and two microphones:

FIG. 16 is a graphic representation showing signal patterns of the data in FIG. 15 after subjected to signal processing in a phase the endotracheal tube remains in normal condition: and

FIG. 17 is a graphic representation showing signal patterns of the data in FIG. 15 after subjected to signal processing in a phase the breathing circuit falls away from the endotracheal tube.

### DESIGNATION OF CODE OR REFERENCE SIGN

1 ventilator

2 ventilator circuit

3 T-shaped connector

4, 10 microphone

5, 25 pressure sensor

6 signal processing circuit

7 personal computer

8 endotracheal tube

9 cuff

11 pilot balloon

12 container for the signal processing circuit

13 adaptive noise canceller

14 low-pass filter

15, 17 short-time Fourier transform (STFT)

16 adaptive line enhancer

18, 19 adaptive filter

20 bacteria filter

22 connector

23 air pump

26 controller unit

28 actuator

30 trachea or airway

### DETAILED DESCRIPTION OF THE PREFERRED EMBODIMENT

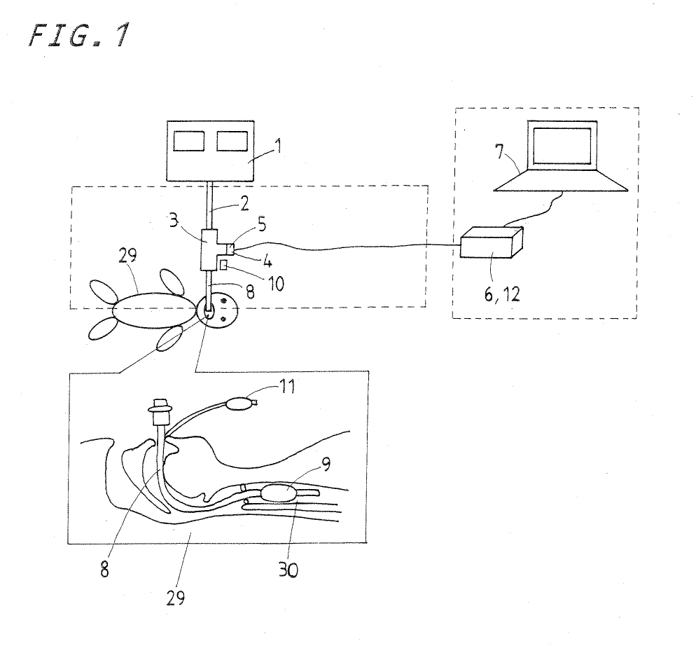

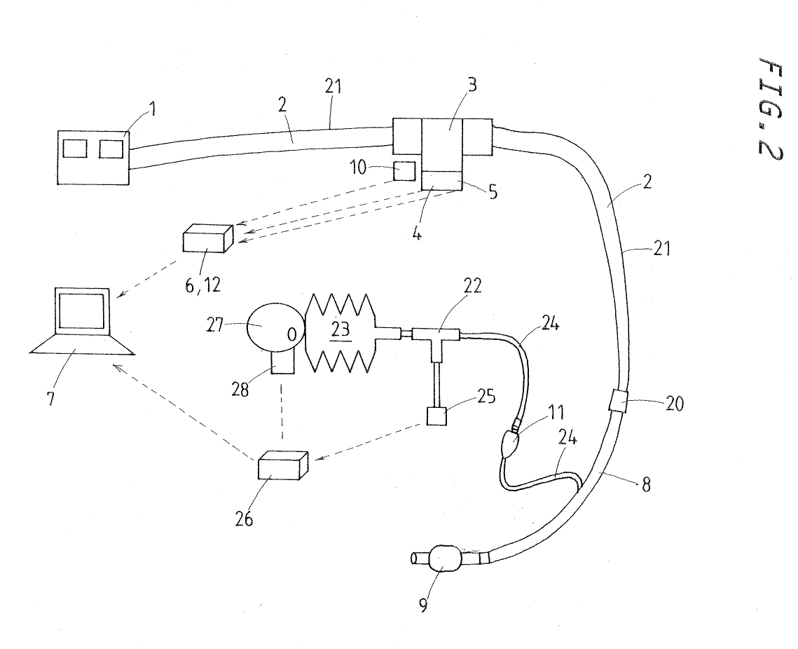

A preferred embodiment of a maintenance and management system for an endotracheal tube according to the present invention will be described hereinafter in connection with the accompanying drawings. The maintenance and management system for the endotracheal tube as shown in FIGS. 1 and 2 has a ventilator circuit 2 of a tube connected with a mechanical ventilator 1, a microphone 4 and a pressure sensor 5, or a first pressure sensor, mounted on the ventilator circuit 2 through a T-shaped connector 3, and another microphone 10 installed nearby the connector 3. An endotracheal tube 8 to be inserted into a trachea or airway 30 of a patient 29 is communicated with the connector 3. Thus, the endotracheal tube 8 may be easily connected to the ventilator 1 through the connector 3 with accompanying both the microphone 4 and the pressure sensor 5. Two sensors, namely the microphone 4 and the pressure sensor 5, issue signals, respectively, which are sent through a signal processing circuit 6 and captured into a personal computer 7 by the help of an A/D converter, in which the incoming signals are processed for diagnostic purpose of associated complications.

With the maintenance and management system for the endotracheal tube constructed as stated earlier, the microphone 4 detects a breath sound and the pressure sensor 5 measures a circuit pressure in the ventilator circuit 2. Both the microphone 4 and the pressure sensor 5 are mounted on one branch of the T-shaped connector 3. Moreover, another microphone 10 is installed nearby the T-shaped connector 3 to detect a noise. Data of detection signals derived from the microphones 4, 10 and pressure sensor 5 are applied to the personal computer 7 via the signal processing circuit 6 which is composed of an amplifier circuit and so on and accommodated in a container 12. The data gets recorded and processed in the personal computer 7. The ventilator circuit 2 is connected with an endotracheal tube 8 which is provided at an end thereof with an inflatable cuff 9 and connected to a pilot balloon 11 through a tube 24. The pilot balloon 11 is further connected through via another tube 24 to a connecter 22 in which there are installed an air pump 23, and a second pressure sensor 25 to measure a pneumatic pressure exerted from the air pump 23. A signal of the second pressure sensor 25 in steady state matches with the cuff pressure and is sent to a controller unit 26 which has a signal processing circuit to control the actuation of the air pump 23 in accordance with the data sent from the second pressure sensor 25. The air pump 23 is actuated with a cam 27, which is driven by an actuator 28 under a command from the controller unit 26 having a signal processing circuit therein. Operating data of the controller unit 26 is sent to the personal computer 7. The signal derived from the second pressure sensor 25 is applied to the controller unit 26, which in turn controls the actuator 28 and sends data obtained as the result of the signal processing to the personal computer 7.

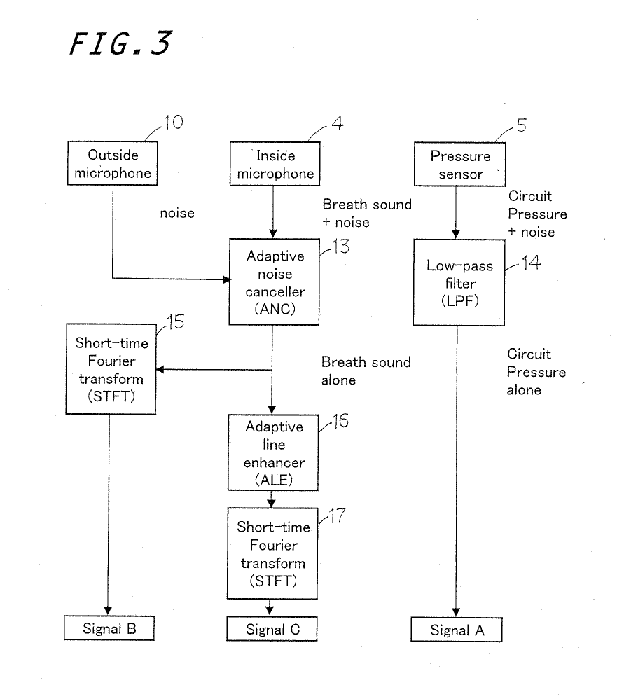

Referring to FIG. 3, there is shown how to process or treat data in the detection signals derived from the microphones 4, 10 and the pressure sensor 5. The signals of the microphones 4 and 10 are sent to an adaptive noise canceller 13. The signal from the microphones 4 is a mixture of a breath sound and a noise, whereas the signal from the microphone 10 has only the noise. The adaptive noise canceller 13 processes the signals of the microphones 4 and 10 to output only the breath sound. The signal from the pressure sensor 5 has a mixture of a circuit pressure and a noise pressure and, therefore, is sent to a low-pass filter 14, which in turn processes the signal to generate a signal A of only the circuit pressure. The breath sound outputted from the adaptive noise canceller 13 is in turn made into a signal B via a short-time Fourier transform 15, or alternatively into a signal C via another short-time Fourier transform 17 after the process at an adaptive line enhancer 16.

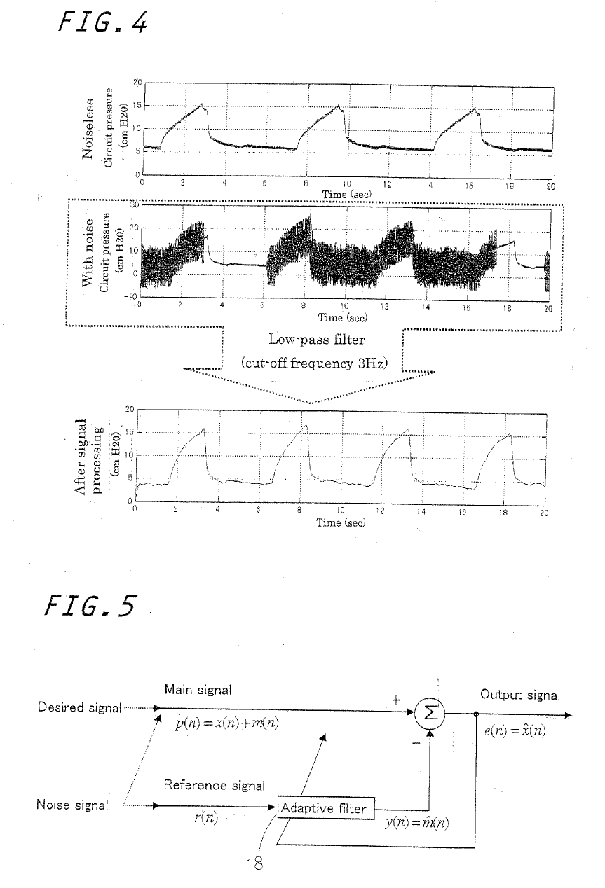

The following some description is the reason and effect about why the output of the pressure sensor 5 is applied to the low-pass filter 14. With the maintenance and management system for the endotracheal tube of the present invention, the pressure inside the breathing circuit is detected with the pressure sensor 5. The signal derived from the pressure sensor 5, if noiseless, would have a regular waveform as shown at top in FIG. 4. In contrast, when the pressure sensor 5 came under the influence of any high frequency noise originating in an electrocautery and so on, the signal from the pressure sensor 5 could get a waveform shown at middle in FIG. 4. To cope with this, a low-pass filter 14 having cutoff frequency of, for example, 3 Hz was introduced into the preferred embodiment of the present invention for reduction of the noise. A signal after passed through the low-pass filter 14 had a waveform like a noiseless signal as shown in bottom in FIG. 4. From this, the low-pass filter 14 has proved successful to reduce the noise. In FIG. 4, an abscissa in coordinates refers to time (seconds) and an ordinate refers to pressure (cmH 20) in the circuit.

The following some description is the reason and effect about why the adaptive noise canceller 13 is adapted to the breath sounds. With the maintenance and management system for the endotracheal tube of the present invention, an experiment was conducted to see how the adaptive noise canceller 13 is effective to decrease the impact of the nearby surrounding noises getting mixed in the breath sounds. For the experiment, there were prepared an endotracheal tube with a balloon at the tip thereof and a large air pump. In the experiment, the noise was a voice mixed in the breath sound. The adaptive noise canceller 13 is said a filter to lower the noise based on two observed signals. Especially, lowering the noise contained in a primary signal was conducted with a noise estimate based on a reference signal. Referring to FIG. 5, there is shown a block diagram of the adaptive noise canceller 13. A sign p(n) denotes the primary signal, x(n) is a desired signal and m(n) is the noise. The primary signal p(n) is equal to the desired signal x(n) plus the noise m(n): p(n)=x(n)+m(n).

By comparison, a reference signal r(n) refers an observed signal originating in the same location as a source of the noise contained in the desired signal. As this occurred, it was assumed that there is no correlation between x(n) and m(n) and also between x(n) and r(n), whereas there is a correlation between m(n) and r(n).

The following is some description about the adaptive filters 18 and 19.

In case where characteristics of the signals or the noises are well known and kept in steady state, the objective of the present invention would be attained with digital filter with fixed filter coefficients. However, the filter design based on the frequency domain has need of spectral value of the signal and noise. The filter designed as stated just above is unsuited in events where the signals are unsteady in their characteristics, the spectral overlap is between the signal and the noise, or the frequency of the signal and/or the noise are unknown. In electromyogram (ECG) measurements of pregnant woman, for example, electric impulses of a fetus and a mother are measured in getting mixed together on ECG and unable to be separated away from each other. To cope with this, there is needed the adaptive filter, which evaluates only the a signal component of the noise, learning the characteristics of the noise signal while getting a filter coefficient varying to be adjusted thereby making it possible to separate the noise from the mixed desired signal.

If an input signal is the reference signal r(n) and the filter coefficient of the adaptive filter 18 is denoted by w(k), an output y(n) of the adaptive filter 18 is represented by the equation (1):

$$y ( n ) = {\sum }_{k = 0}^{M - 1} w ( k ) r ( n - k ) \qquad Formula 1$$

where M is the filter order; w(i) (0≦k<M), the filter coefficients.

The filter coefficient of the filter is updated or renewed according to an adaptive algorithm to lessen an error e(n) between the desired signal x(n) and the output of the filter, self-adjusting to the characteristics of the unsteady-state signals. The adaptive algorithm is to update the coefficient vector in the adaptive filter. Here was used the Least-Mean-Square (LMS) algorithm to update the filter coefficient so as to keep the mean square error E[e(n)2] of the error e(n) in the adaptive filter.

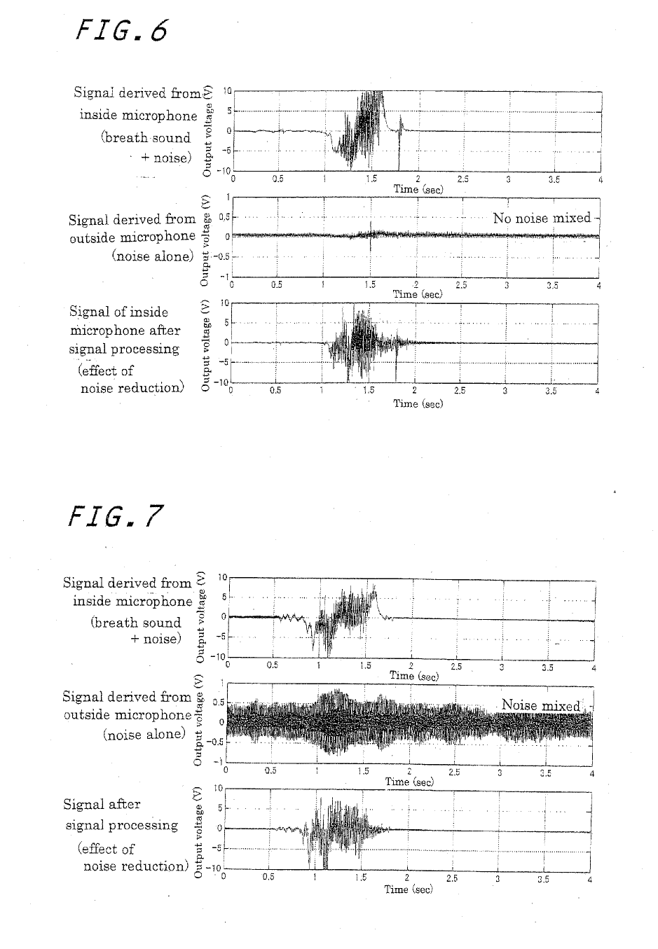

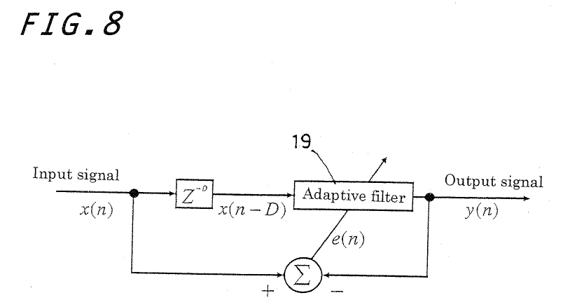

Getting an error between the primary signal and the noise m(n) [=y(n)] estimated in the adaptive filter 18 results in the workings of estimating the desired signal. The noise (m) is shown with hat/caret symbol, when the error signal e(n) becomes the output of the adaptive filter 18. The filter coefficient of the adaptive filter, as being updated with using the error signal e(n), is adjusted adaptively to characteristics of the unsteady-state signals. Thus, the output of the adaptive filter 18 works to estimate the noise included in the primary signal. In FIG. 6, there is shown the result when no noise gets mixed. FIG. 7 shows the result when the noise gets mixed. It is found from the comparison between FIGS. 6 and 7 that the signal of the outer microphone 10 is larger. From this, the outer microphone 10 turns out to have detected only the noise. After comparison between the frequency analyses (not shown) of the results shown in FIGS. 6 and 7 or between the frequency analyses when the noise gets mixed and when no noise gets mixed, it was found that the signals sensed with both the inner and outer microphones 4 and 10 when the noise was mixed contained therein horizontal streaks whose frequency component was considered to be arose out of the voice. The noise reduction was identified as the result of signal processing. Thus, it was found that the adaptive noise canceller 13 could lower the influence of any foreign noise.

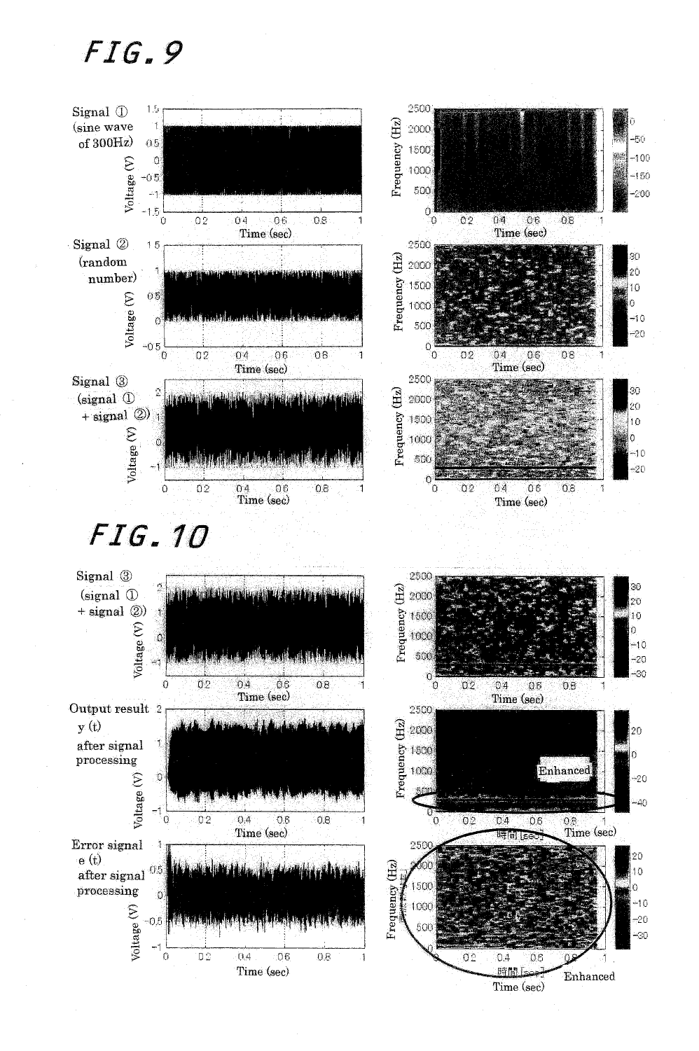

The adaptive line enhancer 16 is a filter used to extract narrowband signals from broadband signals. The filter is called as the line enhancer because of the function as stated earlier. Knowing existing range of the narrowband isn't necessary. Referring to FIG. 6, there is shown a block diagram of the adaptive line enhancer 16. An adaptive filter 19 is the same in input-output relation with the adaptive noise canceller 13, but its input signal or reference signal is a delayed version of the observed signal. The amount of delay is set in a manner that noise components of a delay-before signal and a delay-after signal become irrelevant with each other. The adaptive line enhancer 16 was confirmed to be effective with using a simulated signal. The simulated signal as shown in FIGS. 9 and 10 was a signal resulting from superposition of a random signal on a sinusoidal signal having the frequency of 300 Hz. When the input signal of the simulated signal was processed in the adaptive line enhancer 16, the existence of an output signal y(n) and error signal e(n) was found. As a result as stated earlier, it was said that the existence of the output signal y(n) proved the adaptive line enhancer 16 extracted the narrowband signal and vice versa the existence of the error signal e(n) proved the extraction of the broadband signal. Thus, the adaptive line enhancer 16 was proved effective to the extraction of either of the narrowband and broadband signals.

In preparation for the medical experiments of the maintenance and management system for the endotracheal tube according to the present invention, the microphones 4, 10 and the pressure sensor 5 were easily combined in the ventilator circuit 2 with the aid of the connector 3. The signals originated in the two microphones 4, 10 were captured into the personal computer 7 with the help of an analog-to-digital converter via signal processing circuit 6, and processed to be used for the diagnosis of associated complications. Moreover, the pilot balloon 11 connected to the endotracheal tube 8 having the inflatable cuff 9 at an end thereof was communicated with the pressure sensor 25 and the air pump 23 of the air delivery mechanism through the connector 22. The value measured at the pressure sensor 25 was taken into the controller unit 26 having the signal processing circuit therein and the command issued from the controller unit 25 energized the actuator 28 to drive air pump 23 through the cam 27, thereby controlling the cuff pressure.

With the experiments as stated later carried out in the mechanical ventilation, the breath sounds and the circuit pressures detected at the sensing elements 4, 5 and 10 in the breathing circuit 2 were subjected to the signal processing based on the adaptive filters 18, 19 to make the estimation or prediction of the any complications. The maintenance and management system for tracheal tube embodied as described earlier was identified as working normally on the basis of the experimental results recited below:

a) Estimated breakaway in the breathing circuit; good effect

b) Estimated air leakage; good effect

c) Estimated stenosis in the breathing circuit; good effect to some extent

d) Elimination of foreign noise; good effect

e) Noise reduction when the noise originating in the electrocautery got mixed together; good effect

With the clinical tests whose results will be recited later, the maintenance and management system for tracheal tube constructed according to the present invention was installed in the breathing circuit to measure the sound and pressure picked up or sensed in the breathing circuit in the course of the operation performed really. A breathing apparatus was selected alternatively from the ventilator 1 and the resuscitation bag. There was built in a bacteria filter 20 to trap bacteria and dust in the air captured by the mechanical ventilator 1.

(a) The clinical tests with using the ventilator 1 were conducted on any one of three conditions as stated below:

### Normal State (Normal)

(2) Abnormal state in which the endotracheal tube collapsed in part (abnormal)
(3) Abnormal state in which the cuff pressure decreased to cause air leakage (abnormal)

(b) The clinical tests with using the resuscitation bag were conducted on any one of two conditions as stated below: Normal state (normal)
(2) Abnormal state in which the breathing circuit 2 disengaged from the endotracheal tube, or broke away from the endotracheal tube (abnormal)

The results of the clinical tests as stated earlier were as follows.

(a) Regarding the clinical tests with the ventilator 1

In FIG. 11, there are shown some samples of data derived from three sensing elements.

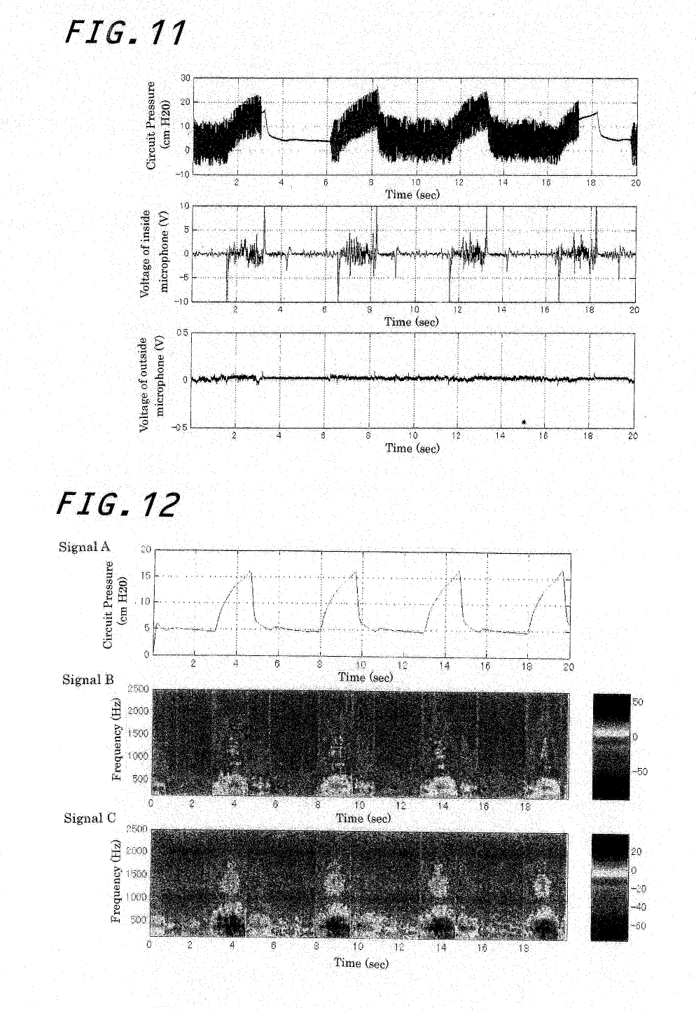

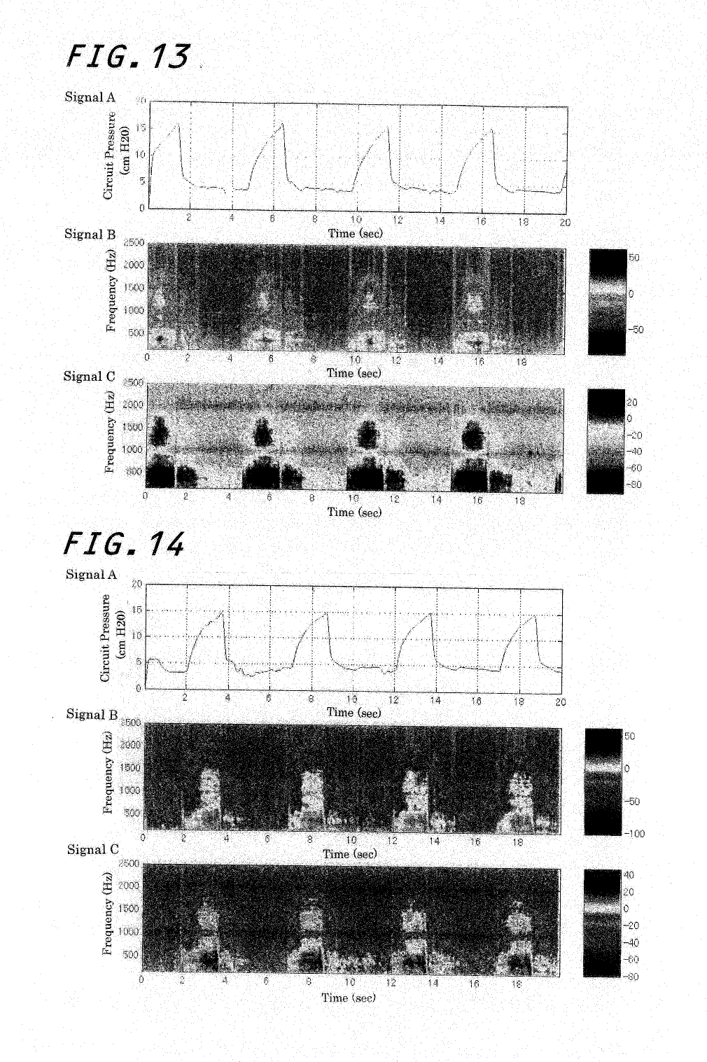

Next, FIGS. 12 to 14 show results after signal processing

The following was ascertained by putting all these together.

1. The peak of the signal A (value of the pressure) in the case of the air leakage was found less by a matter of 1 [cmH 20]. This was considered that the circuit pressure failed to rise up to the pressure level of the normal state because of the air leakage.

2. As for the signal B (breath sound after noise reduction) in the case of air leakage, it was found that the power of frequency, or the value of frequency, rose (especially, 150 Hz˜700 Hz) as the pressure increased. Moreover, it was identified that no increase in the power of frequency was observed in other two states.

3. As for the signal C (breath sound after noise reduction) in the case of the endotracheal tube 8 collapsed partially, the power of frequency not less than 1500 Hz was found much as compared with the normal state.

(b) Regarding the clinical tests with the resuscitation bag

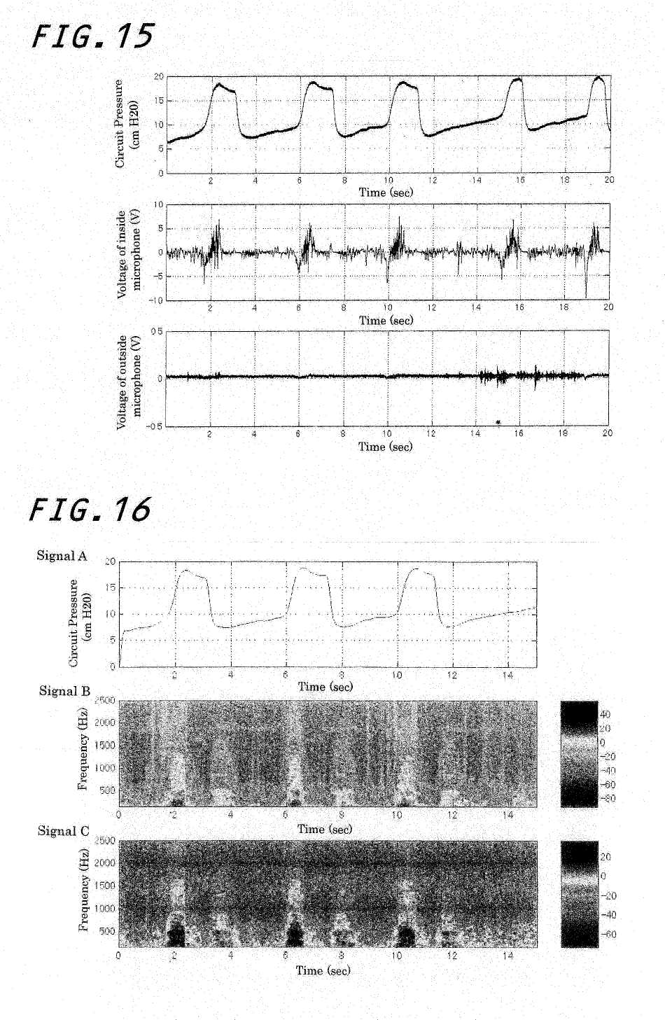

In FIG. 15, there are shown data taken from three sensing elements.

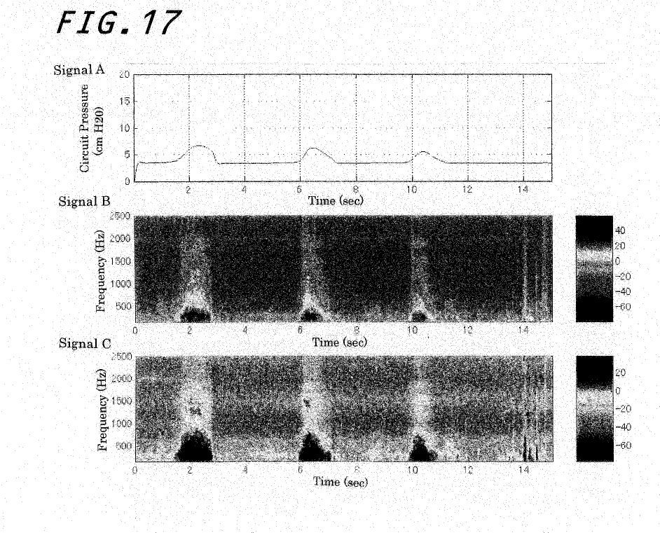

Next, FIGS. 16 to 17 show results after signal processing

The following was ascertained by putting all these together.

The peak of the signal A (value of the pressure) taught that the circuit pressure didn't rise over the normal state.

It was learned from the results of (a) and (b) that observations of the breath sound on the basis of the three signals after subjected to signal processing made it possible to diagnose most of modest complications.

Overall, the complications recited below could be diagnosed with the help of the signals A, B and C:

1. The signal A is successful in diagnosis of the complications including the breakaway of the circuit, and air leakage,

2. The signal B is successful in diagnosis of the complications including air leakage, and

3. The signal C is successful in diagnosis of the complications including the stenosis of the endotracheal tube.

### INDUSTRIAL APPLICABILITY

The maintenance and management system of the endotracheal tube according to the present invention has the industrial applications including the medical practice site, medical education, emergency medicine, and so on, especially the anesthesia, intensive care, first-aid care, and so on and further a diversity of clinical trainings including postgraduate clinical trainings, practices for certified license of intubation to the medical technicians, residency teaching hospitals, training facilities for medical technician, and so on.

What is claimed is:

1. A maintenance and management system for tracheal tube comprising; an endotracheal tube for airway management, an air supply system to force air into a desired area through the endotracheal tube, a tube connecting the endotracheal tube with the air supply system to complete a ventilator circuit, a microphone to sense a breath sound transmitted via the endotracheal tube, a first pressure sensing element to measure a circuit pressure in the ventilator circuit, the microphone and first pressure sensing element being built in the ventilator circuit tube, a personal computer receiving data derived from the microphone and first pressure sensing element and processing the data therein, the personal computer measuring whether the breath sound sensed with microphone is within a desired value range and then signing the data if within the desired value range while issuing abnormality output signal instead of the data if over the desired value range, and the personal computer further measuring whether the circuit pressure detected with the first pressure sensing element is within a desired value range and then signing the data if within the desired value range while issuing abnormality output signal instead of the data if over the desired value range;
an adaptive noise canceller comparing a noise other than the breath sound sensed with the microphone to a signal detected with an outer microphone installed nearby the microphone and outside of the ventilator circuit, then excluding the noise; and
an adaptive line enhancer extracting a narrowband signal from a broadband signal which includes only the breath sound outputted from the adaptive noise canceller.

2. A maintenance and management system for tracheal tube according to claim 1, which further has a low-pass filter to exclude a noise other than the circuit pressure included in a signal sensed with the first pressure sensing element.

3. A maintenance and management system for tracheal tube according to claim 1, wherein the personal computer, when the data detected with the microphone and the first pressure sensing element correspond to the abnormality output signal, measures whether what complication is taught based on the data, displaying and warning the complication.

4. A maintenance and management system for tracheal tube according to claim 3, wherein the personal computer measures and warns the complication to be an air leak occurring in the endotracheal tube, in response to an abnormal frequency power of the breath sound after noise reduction of the signal derived from the microphone.

5. A maintenance and management system for tracheal tube according to claim 3, wherein the personal computer measures and warns the complication to be a stenosis occurring in the endotracheal tube, in response to an abnormal frequency power of the breath sound after signal processing of the signal derived from the microphone.

6. A maintenance and management system for tracheal tube according to claim 1, which further has a cuff-pressure regulator connected to the endotracheal tube through a pilot balloon to regulate a cuff pressure in a cuff attached to the endotracheal tube, and a second pressure sensing element to gauge the cuff pressure, and wherein the cuff-pressure regulator controls a pneumatic pressure applied to the cuff in response to a data originating in the second pressure sensing element, keeping and regulating the cuff pressure at a prescribed pressure.

7. A maintenance and management system for tracheal tube according to claim 6, wherein the personal computer measures whether the cuff pressure gauged with the second pressure sensing element is in normal state or abnormal state, keeping the state detected with the second pressure sensing element if in normal state, and instead warning occurrence of the complication if in abnormal state.

8. A maintenance and management system for tracheal tube according to claim 6, wherein the personal computer in response to the abnormal cuff-pressure gauged with the second pressure sensing element measures and warns the complication to be an air leak caused by a cuff-pressure reduction, aberrant aspiration of foreign body including digestive juice, blood and so on into the airway, or an oppressive pressure against a blood flow in a tracheal mucosa.
